(19) United States
(12) Patent Application Publication
(10) Pub. No.: US 2020/0197227 A1
(43) Pub. Date: Jun. 25, 2020

(54) POST-OPERATIVE SURGICAL WOUND DRESSING

(71) Applicants: KCI USA, INC., San Antonio, TX (US); KCI LICENSING, INC., San Antonio, TX (US); SYSTAGENIX WOUND MANAGEMENT, LIMITED, West Sussex (GB)

(72) Inventors: Christopher B. LOCKE, Bournemouth (GB); Leanna POOLE, Rawtenstall (GB); Thomas LANE, Skipton (GB); William PIGG, Elvington (GB); Craig DELURY, Gargrave (GB); Kyle TURTON, Skipton (GB); Deborah ADDISON, Near Clapham (GB); Richard COULTHARD, Verwood (GB)

(21) Appl. No.: 16/615,346

(22) PCT Filed: May 21, 2018
(86) PCT No.: PCT/US2018/033634

§ 371 Date: Nov. 20, 2019

Related U.S. Application Data
(60) Provisional application No. 62/509,573, filed on May 22, 2017.

(51) Int. Cl. A61F 13/02; A61F 13/00; A61M 1/00; A61L 26/00; A61L 15/22

(52) U.S. Cl. CPC A61F 13/0206; A61F 13/00068; A61F 13/0246; A61F 2013/00719; A61L 26/008; A61L 15/22; A61F 2013/00634; A61M 1/0088

(57) ABSTRACT

Described herein is a wound dressing comprising: a composite island configured to collectively exhibit substantially elastic recovery under wound treatment conditions and comprising a foam layer and an absorbent layer coupled to the foam layer, the foam layer exhibiting substantially elastic recovery under wound treatment conditions and the absorbent layer not exhibiting substantially elastic recovery under wound treatment conditions; a backing layer having a margin that extends beyond the composite island; and an adherent layer disposed on at least the margin of the backing layer. In addition, described herein is a method of eliminating, minimizing, or reducing edema for a wound surrounded by tissue, by positioning the wound dressing over the wound, optionally such that at least a portion of the adherent layer contacts the tissue.

### CROSS-REFERENCE TO RELATED APPLICATIONS

This application claims the benefit of priority to U.S. Provisional Application No. 62/509,573, filed on May 22, 2017, which is incorporated herein by reference in its entirety.

### TECHNICAL FIELD

The present technology relates to compositions and devices, including wound dressings, for example, for application to a tissue site such as a wound.

### BACKGROUND

A wide variety of materials and devices, generally characterized as “wound dressings,” are generally known in the art for use in treating an injury or other disruption of tissue. Such wounds may be the result of trauma, surgery, or disease, and may affect skin or other tissues. In general, wound dressings may control bleeding, absorb wound exudate, ease pain, assist in debriding the wound, protect wound tissue from infection, or otherwise promote healing and protect the wound from further damage.

Although the clinical benefits and advantages of wound dressings may be widely accepted, improvements to wound dressings may benefit healthcare providers and patients.

### BRIEF SUMMARY

New and useful compositions of dressing layers and wound dressings including such dressing layers, methods for manufacturing same, and methods for using the same are set forth in the appended claims. Illustrative embodiments are also provided to enable a person skilled in the art to make and use the claimed subject matter.

Some examples may comprise a backing layer, an adherent layer disposed on at least a margin of the backing layer, and a composite island configured to exhibit substantially elastic recovery. The composite island may comprise, for example, a foam layer coupled to an absorbent layer. The foam layer may have substantially elastic recovery under wound treatment conditions, and the absorbent layer may not have substantially elastic recovery under wound treatment conditions. The backing layer may extend beyond the composite island to form a backing layer margin around the composite island.

In some examples, a wound dressing may comprise a backing layer, a perforated envelope, and an absorbent layer surrounded by the perforated envelope. The perforated envelope can preferably be configured to exhibit substantially elastic recovery and may be oriented such that the backing layer extends beyond the perforated envelope, whereby a backing layer margin remains. An adherent layer may be disposed on at least the margin of the backing layer.

Other examples of a wound dressing may comprise a backing layer, a composite island, and an adherent layer disposed on at least a margin of the backing layer and extending over a margin of the composite island, thereby fastening the adherent layer to the margin of the composite island. The composite island may be configured to exhibit substantially elastic recovery and can comprise a foam layer coupled to an absorbent layer. The foam layer may preferably have substantially elastic recovery under wound treatment conditions. The absorbent layer may be substantially inelastic under wound treatment conditions. A plurality of fenestrations in the absorbent layer may be collectively configured to mitigate plastic deformation of the absorbent layer.

Another example of a wound dressing may comprise an adherent layer disposed between the absorbent layer and the backing layer, such that the adherent layer extends beyond the absorbent layer to form an adherent margin. In this example, each of the plurality of fenestrations can extend inward from an edge of the absorbent layer no further than a midpoint between opposing edges, so as to form a central backbone with appendages extending outward therefrom. In some examples, at least a majority of the plurality of fenestrations may be oriented to form an acute angle between each fenestration and a width or length direction.

Another example relates to a method of eliminating, minimizing, or reducing edema, such as post-operative edema, for a wound surrounded by tissue. The method may comprise positioning a wound dressing described herein over the wound, such that at least a portion of the adherent layer of the wound dressing contacts the tissue.

Objectives, advantages, and one or more preferred modes of making and using the claimed subject matter may be understood by reference to the accompanying drawings, in conjunction with the following detailed description of illustrative embodiments.

### DRAWINGS

FIG. 1 is a perspective view in cross-section of a wound dressing according to this specification.

FIG. 2 is a diagram of the wound dressing of FIG. 1 with a therapy system.

FIG. 3 is a diagram of an example of the wound dressing of FIG. 1 having a central backbone with appendages extending outward therefrom.

FIGS. 4A-4C are diagrams of an example of the wound dressing of FIG. 1.

FIG. 4A is a top view of an example having a fenestrated absorbent layer. FIG. 4B is a section view of the wound dressing of FIG. 4A taken along line 4B. FIG. 4C illustrates the wound dressing of FIG. 4A stretched in the length direction while the length of the fenestrations are in the width direction.

FIGS. 5A-5C are diagrams of another example of the wound dressing of FIG. 1. FIG. 5A is a top view of an example having a plurality of fenestrations of several different dimensions. FIG. 5B is a section view of the wound dressing of FIG. 5A taken along line 5B. FIG. 5C illustrates the wound dressing of FIG. 5A stretched in the length direction while the length of the fenestrations are in the width direction.

FIGS. 6A-6C are diagrams of another example of the wound dressing of FIG. 1. FIG. 6A is a top view of an example having a central composite island with a foam layer coupled to an absorbent layer that is absent in at least a portion of a central zone of its length. FIG. 6B is a section view of the wound dressing of FIG. 6A taken along line 6B. FIG. 6C illustrates the wound dressing of FIG. 6A stretched in the length direction.

FIGS. 7A-7C are diagrams of another example of the wound dressing of FIG. 1. FIG. 7A is a top view of an example having a plurality of fenestrations and perforations in an absorbent layer. FIG. 7B is a section view of the wound dressing of FIG. 7A taken along line 7B. FIG. 7C illustrates the wound dressing of FIG. 7A stretched in the length direction while the length of the fenestrations are in the width direction.

FIG. 8A is a perspective view of an example of a composite island layer that may be associated with the wound dressing of FIG. 1, FIG. 8B is a diagram of the composite island layer of FIG. 8A in a wound dressing.

FIG. 9A is a perspective view of an example of an absorbent layer that may be associated with the wound dressing of FIG. 1. FIG. 9B is a perspective view of an example of a composite island layer having a foam layer coupled to the absorbent layer of FIG. 9A.

FIGS. 10A-10B are perspective views of two embodiments of composite island layers that may be associated with the wound dressing of FIG. 1.

It should be noted that the figures set forth herein are intended to exemplify the general characteristics of materials and methods among those of the present invention, for the purpose of the description of certain embodiments. The figures may not precisely reflect all the characteristics of any given embodiment, and are not necessarily intended to define or limit specific embodiments within the scope of this invention.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

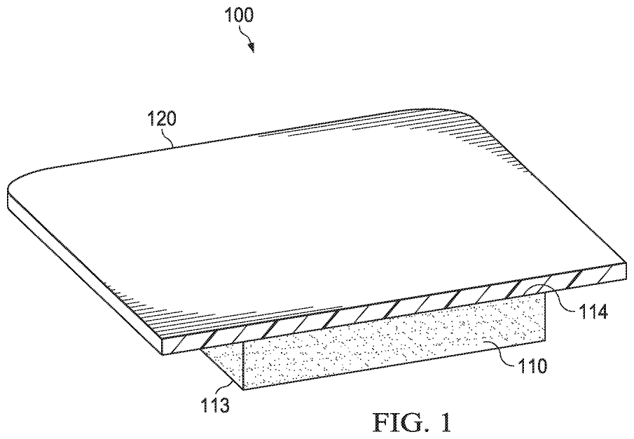

Disclosed herein are embodiments of a dressing layer, embodiments of wound dressings including such a dressing layer, and embodiments of therapy systems including same. Also disclosed herein are embodiments of methods related to (for example, methods of making, methods of using, etc.) the disclosed dressing layers, wound dressings, and therapy systems. For example, FIG. 1 illustrates an embodiment of a wound dressing 100. Generally, and as will be disclosed herein, the wound dressing 100 may be configured to provide therapy to a tissue site in accordance with the disclosure of this specification.

As used herein, “tissue site” is intended to broadly refer to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), skin flaps, and grafts, for example. The term “tissue site” may also refer to areas of any tissue that are not necessarily wounded or defective, but are instead areas in which it may be desirable to add or promote the growth of additional tissue, such as granulation. As noted above, the tissue site can refer to an area of tissue where prophylactic treatment is desired, even without a wound or defect, for example with respect to pressure ulcer prevention.

In some embodiments, a wound dressing may include one or more dressing layers configured to interface with a tissue site. For example, in the embodiment of FIG. 1, the wound dressing 100 may include a dressing layer 110. The dressing layer 110 may generally be configured to be positioned on, over, in, adjacent to, or otherwise in contact with (collectively, “near”) the tissue site.

### Dressing Layer

In various embodiments, the dressing layer 110 may be configured so as to be in contact with a portion of the tissue site, substantially all of the tissue site, or the tissue site in its entirety. If the tissue site is a wound, for example, the dressing layer 110 may partially or completely fill the wound, or may be placed near the wound. In various embodiments, the dressing layer 110 may take many forms, and may have many sizes, shapes, or thicknesses depending on a variety of factors, such as the type of treatment being implemented or the nature and size of a tissue site. For example, the size and shape of the dressing layer 110 may be adapted to the contours of deep and irregular shaped tissue sites, may be configured so as to be adaptable to a given shape or contour, or both. Moreover, in some embodiments, any or all of the surfaces of the dressing layer 110 may comprise projections or an uneven, course, or jagged profile that can, for example, induce strains and stresses on a tissue site, which may be effective to promote granulation at the tissue site.

In some embodiments, the dressing layer 110 may be in substantially sheet form. For example, the dressing layer 110 may comprise a generally planar structure having two opposite-facing planar surfaces and a depth or thickness orthogonal to the planar surfaces. More particularly, for example, the dressing layer 110 may comprise a first surface 113 opposite a second surface 114. The first surface 113 may be adapted to contact a tissue site, having a surface area sufficient to cover an appropriate portion, if not all, of the tissue site. For example, a surface area from about 1 cm² to about 4000 cm² may be suitable for many applications. In various embodiments, the first surface 113 and the second surface 114 may have any suitable shape, examples of which include, but are not limited to, triangles, squares, rhombuses, rhomboids, diamonds, rectangles, trapezoids, ellipses, ellipsoids, circles, semi-circles, pie-wedges, ovals, and various polygons having four, five, six, seven, eight, or more sides. These shapes may additionally or alternatively be adaptations of such common shapes. In some embodiments, shapes with typically rounded edges may be altered to be flatter, such as a rounded hexagonal/octagonal shape made by flattening the rounded edges of a circle. Additionally or alternatively, shapes with typically rounded edges may be altered to be sharper, such as a tear-drop shape made by sharpening a rounded end of an ellipse or ellipsoid, or such as an eye shape made by sharpening two rounded, opposing ends of an ellipse or ellipsoid. Further additionally or alternatively, shapes with typically pointed edges may be altered to be more rounded, such as for a blunt-ended triangle. Still further additionally or alternatively, shapes with typically flat edges may be altered to be more rounded, such as by converting the flat sides of any regular polygon to a sinusoidal edge to form a doily shape with an undulating, curvy edge. The shape and area of the second surface 114 may be customized to the location and type of tissue site onto which the wound dressing 100 is to be applied.

### Dressing Layer Composition

There may be various embodiments of the composition of the dressing layer 110. In some embodiments, the dressing layer 110 may be a single layer, whereas, in some embodiments, the dressing layer 110 may represent a multi-layer composite structure. For example, the dressing layer 110 may comprise at least two layers coupled to each other. In other embodiments, the dressing layer 110 may represent a multi-layer structure where certain layers may be operably linked together, even though some or all may not be strictly coupled to one or more of the other layers. For example, a sandwiched layer can be enveloped within a sealed container layer, in which case neither layer is coupled to the other, but the sandwiched layer can be operably linked to the sealed container layer because of the envelopment. For example, the sandwiched layer may not expand without the sealed container layer also expanding, despite a lack of layer coupling.

In some embodiments, the dressing layer 110 can be configured to exhibit substantially elastic recovery under wound treatment conditions. In some embodiments, the wound dressing 100 can be configured to exhibit substantial elastic recovery under wound treatment conditions. In various embodiments, however, at least one of the component layers of the wound dressing 100 may not exhibit substantially elastic recovery under wound treatment conditions. For instance, in some embodiments, the dressing layer 110 may include a composite island structure, which may comprise an elastic layer, such as a foam layer or an extensible non-woven layer, coupled to an absorbent layer that is not elastic. In this way, the elastic layer can enable the composite island, and thus the wound dressing 100, to exhibit substantially elastic recovery at wound treatment conditions, which may be a desirable characteristic, particularly if the absorbent layer is capable of absorbing generous amounts of aqueous medium, such as saline or wound exudate.

In this context, a material that exhibits substantially elastic recovery under wound treatment conditions is termed “elastic”, and a material that does not exhibit substantially elastic recovery under wound treatment conditions is termed “not elastic”. In some embodiments, the wound dressing 100 or any layer(s) thereof such as the dressing layer 110, having a width and a length perpendicular to the width, may be considered elastic if it exhibits at most about 10%, advantageously about 5% or less or about 2% or less, permanent deformation when subjected to about 50% strain, relative to the length, for about 3 days at that strain level, using an Instron™ mechanical testing machine, for example, at an initial imposed strain rate of about 1% elongation per second up to the total strain value, at which point it can be held for the total strain time. Additionally or alternatively, in some embodiments, the wound dressing 100 or any layer(s) thereof such as the dressing layer 110, having a width and a length perpendicular to the width, may be considered elastic if it exhibits at most about 5%, advantageously about 2% or less or about 1% or less, permanent deformation when subjected to about 25% strain, relative to the length, for about 24 hours at that strain level, using an Instron™ mechanical testing machine, for example, at an initial imposed strain rate of about 10% elongation per minute up to the total strain value, at which point it can be held for the total strain time. Additionally or alternatively, in some embodiments, the wound dressing 100 or any layer(s) thereof such as the dressing layer 110, having a width and a length perpendicular to the width, may be considered elastic if it exhibits at most about 1%, and advantageously about 0%, permanent deformation when subjected to about 10% strain, relative to the length, for about 10 minutes at that strain level, using an Instron™ mechanical testing machine, for example, at an initial imposed strain rate of about 1% elongation per minute up to the total strain value, at which point it can be held for the total strain time. Instron™ testing may be performed at room temperature, such as ˜20-25° C., and at low relative humidity, for example ˜40% RH or less. Even though particular values are specified with respect to the strain test, materials, layers, or compositions may be considered elastic if optionally tested with one or more parametric deviations, including but not limited to: being conducted at a greater strain than specified (for example, between about 50% strain and about 100% strain, between about 25% strain and about 75% strain, or between about 10% strain and about 50% strain), relative to the length; being conducted for a longer time than specified (for example, between about 10 minutes and about 120 hours, between about 24 hours and about 96 hours, or between about 3 days and about 7 days) at the total strain level; and being conducted at an initial strain rate greater specified (for example, between about 1% elongation per minute and about 600% elongation per minute, between about 10% elongation per minute and about 1200% elongation per minute, or between about 1% elongation per second and about 40% elongation per second).

In other example embodiments, the dressing layer 110 may be a single absorbent layer having a plurality of fenestrations configured to mitigate plastic deformation of the single absorbent layer. For example, each of the plurality of fenestrations can extend inward from an edge of the absorbent layer to a midpoint between opposing edges. When the fenestrations extend only part of the way to the midpoint, they can form the apparent shape of a central backbone with appendages extending outward therefrom. Also in such embodiments, the wound dressing 100 may advantageously include an adherent layer coupled to the single absorbent layer, in order to provide an elastic layer boundary, such that the coupled adherent layer can assist the single fenestrated absorbent layer in behaving as if elastic.

Substantial elastic recovery may be particularly important for treating tissue in areas of relatively high articulation or flexure, such as proximal to shoulder, elbow, knee, ankle, or hip joints (particularly knee or elbow joints). It may be desirable for the wound dressing 100 to be able to undergo significant local flexure and substantially retain its shape, and its contact with the tissue site, upon significant articulation. Maintaining shape or tissue contact can reduce the frequency of necessitated dressing changes, reduce tissue blistering from improper contact, increase exudate absorption, reduce wound healing time, increase patient comfort, or combinations thereof.

It should be noted that, even though the mitigation of plastic deformation is described herein as being linked to a plurality of fenestrations, it is possible and contemplated that a single fenestration, if large enough and appropriately located, could acceptably result in mitigation of plastic deformation in the layer that is not elastic, under certain conditions.

An absorbent layer is typically present in the dressing layer 110, either as the single layer or as one of multiple layers. For example, the dressing layer 110 may comprise a composite island having one or more absorbent layers. The absorbent layer may comprise a non-woven material of predominantly non-woven fibers such as gelling fibers, in some embodiments. For example, in various embodiments, the absorbent layer may comprise from about 45 parts to about 100 parts by weight of cellulosic (for example, cellulose ether) fibers and optionally up to about 55 parts by weight of reinforcing fibers. In particular embodiments, the absorbent layer may comprise from about 45 parts to about 95 parts by weight, from about 45 parts to about 90 parts by weight, from about 50 parts to about 90 parts by weight, from about 60 parts to about 90 parts by weight, from about 65 parts to about 85 parts by weight, or from about 70 parts to about 90 parts by weight of cellulosic fibers and about 55 parts to about 10 parts by weight, from about 50 parts to about 10 parts by weight, from about 45 parts to about 10 parts by weight, from about 40 parts to about 10 parts by weight, from about 35 parts to about 15 parts by weight, from about 30 parts to about 10 parts by weight, from about 30 parts to about 15 parts by weight, or from about 25 parts to about 10 parts by weight of reinforcing fibers. In some optional embodiments, biodegradable components may additionally be present in the absorbent layer, for example in amounts from about 1 part to about 20 parts by weight, such as from about 1 part to about 15 parts by wcight or from about 1 part to about 10 parts by weight.

In some multi-layer embodiments, a surface of the absorbent layer not coupled to the elastic layer can be oriented to be a lower or contact layer, whereas, in other multi-layer embodiments, a surface of the elastic layer not coupled to the absorbent layer can be oriented to be a lower or contact layer. In many embodiments, one or more of the elastic layers may comprise or be a foam layer. In some embodiments, it may be advantageous for the absorbent layer to be separated from a tissue site, for example, such that the surface of the elastic layer not coupled to the absorbent layer can be oriented to be a lower or contact layer.

In many embodiments, an absorbent layer may be fenestrated. For example, fenestrations may be slits, usually fully through the thickness of the absorbent layer. The fenestrations can be configured to allow expansion of the absorbent layer upon an applied strain to the wound dressing 100, in some embodiments without significant additional plastic deformation to the fibers in that layer, while coupling to an elastic layer can enable the composite of the two layers to collectively elastically recover upon relief from the applied strain. Thus, in some embodiments, at least a majority of the fenestrations (and particularly at least 90% of the fenestrations) may have their slit length oriented roughly perpendicular to the direction in which the strain is likely to be applied. For example, a majority of the fenestrations may have their slit length oriented parallel to the width, when strain is likely to be applied in the length direction. In additional or alternative embodiments, at least a majority of the fenestrations (and particularly at least 90% of the fenestrations) may have their slit length oriented at an acute angle to the direction in which the strain is likely to be applied. In some such embodiments, the acute angle may be from about 10° to about 80°, from about 20° to about 70°, or from about 30° to about 60°.

In some multi-layer embodiments, the dressing layer 110 may comprise at least two layers that are operably linked together without being strictly coupled, with one layer being a not elastic absorbent layer and another layer being an elastic envelope configured to allow fluid to access the absorbent layer. For example, the envelope may have a macro-structure or micro-structure allowing relatively easy diffusion or fluid flow into the absorbent layer. Additionally or alternatively, the envelope may be perforated to enhance fluid flow into the absorbent layer. In such multi-layer wound dressing embodiments, the elastic recovery of the envelope can be imposed upon the absorbent layer, without directly coupling the envelope to the absorbent layer, and without which envelopment the absorbent layer would not exhibit substantial elastic recovery, upon imposition and release of an applied strain. Additionally or alternatively, in such multi-layer wound dressing embodiments, the envelopment of the absorbent layer can include additional volume not usually present with layers directly coupled together, enabling relatively high levels of absorption of fluid by the absorbent layer with additional degrees of freedom for absorptive expansion.

If cellulosic fibers are present in the absorbent layer, the cellulosic fibers may be composed of at least one of carboxymethyl cellulose (CMC), carboxylethyl cellulose, hydroxyethyl cellulose, hydroxypropyl cellulose, hydroxypropylmethyl cellulose, and cellulose ethyl sulphonate (CES) (particularly carboxymethyl cellulose), for example. In some embodiments, the cellulosic component may be at least partially in a salt form, for example, comprising a physiologically acceptable cation such as sodium. CMC is commercially available from a variety of sources, such as under the tradenames Walocel™ (sold by The Dow Chemical Company) and Cekol® (sold by CP Kelco). If reinforcing fibers are present in the absorbent layer, for example, the reinforcing fibers may be composed of at least one of a polyurethane gel, an amide polymer such as Nylon 6,6, an olefin polymer such as HDPE, an ester polymer such as PET, and a modified acrylamide polymer. If biodegradable components are present in the absorbent layer, for example, the biodegradable components may be composed of, but not limited to, an alginic acid, an alginate salt, chitosan, chitin, a guar gum, a locust bean gum, a xanthan gum, a karaya gum, gelatin, pectin, a starch derivative, a glycosaminoglycan, a galactomannan, a chondroitin salt, heparin, a heparin salt, collagen, oxidized regenerated cellulose (ORC), hyaluronic acid, a hyaluronate salt, or a combination thereof. For such listed salt components, the salt components may include any reasonable counterions, such as sodium, calcium, ammonium, or the like, or combinations thereof. The biodegradable component(s) can be, for example, in the form of a film or a foam, such as an open-cell foam, a reticulated foam, or combinations thereof. If in foam form, the average pore size may vary according to needs of a prescribed therapy, for example, from about 400 microns to about 600 microns). Other physico-chemical properties of biodegradable components, such as tensile strength, may be chosen or manipulated, for example, to be suitable to needs of a prescribed treatment.

In some embodiments, particularly if biodegradable components are included, the dressing layer 110 may be characterized as having some biodegradable character or as exhibiting biodegradability. “Biodegradable” and “biodegradability” may individually or collectively refer to a characteristic of a material to disintegrate, degrade, or dissolve upon exposure to physiological fluids or processes, for example, when the dressing layer 110 is positioned with respect to a tissue site. For example, in some embodiments, the dressing layer 110 or a material from which the dressing layer 110 is formed may form a gel when contacted with an aqueous medium, such as water, saline, blood, or wound exudate. Such biodegradability may be exhibited as a result of chemical process or condition, a physical process or condition, or some combination thereof. For example, the biodegradable characteristics of the dressing layer 110 may substantially reduce or eliminate the need to remove the dressing layer 110 from a tissue site to which it is applied. In some embodiments, at least about 90% by weight of the biodegradable component (particularly at least about 95% by weight, at least about 99% by weight, or about 100% by weight) may be disintegrated, degraded, or dissolved with in a time period of from about 15 days to about 24 hours (particularly from about 12 days to about 36 hours or from about 10 days to about 48 hours), from introduction into a physiological environment when incubated with simulated physiological fluid at a temperature of about 37° C.

### Optional Dressing Layer Additives

In some embodiments, the wound dressing 100, and particularly the dressing layer 110, may optionally comprise one or more additional materials. Such optional components may include, for example, active materials such as preservatives, stabilizing agents, plasticizers, matrix strengthening materials, dyestuffs, and combinations thereof.

Additionally or alternatively, the wound dressing 100, and particularly the dressing layer 110, may comprise one or more additional active materials, for example, antimicrobial agents that may be effective to aid in wound healing. Non-limiting examples of such active materials may include non-steroidal anti-inflammatory drugs such as acetaminophen, steroids, antimicrobial agents such as penicillins or streptomycins, antiseptics such as chlorhexidine, growth factors such as fibroblast growth factor or platelet derived growth factor, and other well-known therapeutic agents, alone or in combination. If present, such active materials may typically be included at any effective level that show therapeutic efficacy, while preferably not being at such a high level as to significantly counteract any critical or desired physical, chemical, or biological property of the wound dressing.

Depending upon the therapeutic goal(s), the active material(s) may be loaded at a level of from about 10 wppm to about 10 wt % of the layer(s) in which it(they) are present, for example, from about 50 wppm to about 5 wt % or from about 100 wppm to about 1 wt %.

In some embodiments, the antimicrobial agents may comprise a safe and effective amount of poly(hexamethylene biguanide) (“PHMB”), which is also known as polyaminopropyl biguanid (“PAPB”) and polyhexanide, having the following general formula.

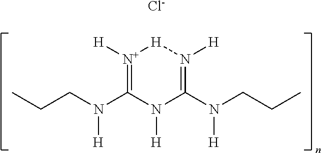

PHMB can be a cationic broad spectrum antimicrobial agent. PHMB may be synthesized by a variety of methods, including polycondensation of sodium dicyanamide and hexamethylenediamine. PHMB is commercially available from a variety of sources. In some embodiments, the PHMB may be present in one or more of the dressing layers at a level of from about 0.005 wt % to about 0.025 wt % of each layer in which it is present, particularly from about 0.007 wt % to about 0.2 wt % or from about 0.008 wt % to about 0.012 wt %, or in some cases at about 0.01 wt %. In some embodiments, the PHMB may be present in one or more of the dressing layers at a level of from about 0.05 wt % to about 3 wt % of each layer in which it is present, particularly from about 0.1 wt % to about 2.5 wt %, from about 0.3 wt % to about 2 wt %, from about 0.5 wt % to about 1.5 wt %, or in some cases at about 1 wt %. In alternative embodiments, silver compounds having antimicrobial efficacy may completely or partially replace the PHMB, as desired.

In some embodiments where CMC is not already present, the composition may comprise CMC as a modifier for one or more characteristics of the wound dressing or dressing layer(s), for example, the rheological, absorbency, and other structural characteristics. CMC may be present in the layer(s) at any level appropriate to result in the desired absorbency, rheological, or other structural characteristics of the wound dressing.

In some embodiments, the dressing layer 110 may contain a strengthening material, which can improve the handling characteristics of the wound dressing 100, for example, by decreasing its susceptibility to tearing. The strengthening material may comprise non-gelling cellulose fibers in some examples. Such “non-gelling” cellulose fibers may be substantially water insoluble and may be produced from cellulose that has not been chemically modified to increase water solubility, for example, as contrasted from carboxymethyl cellulose or other cellulose ethers. Non-gelling cellulose fibers are commercially available, such as under the tradename TENCEL (sold by Lenzing AG). In some embodiments, such fibers may be processed from a commercially-available continuous length, by cutting into lengths from about 0.5 to about 5 cm or from about 2 to about 3 cm in length. The non-gelling cellulose fibers may be present in the composition at any level appropriate to result in the desired physical characteristics of the composition. In general, when present, the non-gelling cellulose fibers may comprise from about 1% to about 55% of the layer by weight, particularly from about 5% to about 40% of the layer by weight or from about 10% to about 25% of the layer by weight. In some embodiments, if present, the non-gelling cellulose fibers can be characterized as an additional or alternative reinforcing fiber and can be present in reinforcing fiber amounts.

### Wound Dressing—Cover (Backing Layer)

In most embodiments, aside from dressing layer 110, the wound dressing 100 may comprise one or more additional layers. In various embodiments, such additional layers may perform any of a variety of functions including, for example, adherence of the wound dressing 100 to a tissue site or to surrounding tissues, increasing structural rigidity of the wound dressing 100, protection from moisture or other contaminants in the external environment, protection of a wound surface, delivery of one or more active or other materials to the wound surface, or combinations thereof. In various embodiments, the additional layers may be conformable to a wound surface or to the surrounding tissues, for example, being capable of conforming such that the wound-facing surfaces of the wound dressing 100 are in substantial contact with a tissue site.

For example, in the embodiment of FIG. 1, the wound dressing 100 comprises a backing layer 120, which may be positioned over the dressing layer 110, for example, so as to cover the dressing layer 110 at a tissue site. The backing layer 120 may have a first surface and a second surface. The first surface of the backing layer 120 may face a wound when applied, and can support the dressing layer 110, for example, such that a surface of the dressing layer 110 can be proximate to the first surface of the backing layer 120. In some embodiments, the surface of the dressing layer 110 may be in contact with and adhered to the first surface of the backing layer 120.

In particular embodiments, the backing layer 120 of the wound dressing 100 may extend beyond the boundaries or edges of the dressing layer 110, so as to exhibit an exposed backing layer margin, which may typically be exhibited on the second surface of the backing layer 120.

In some embodiments, the backing layer 120 may generally be configured to provide a barrier to microbes, a barrier to external contamination, and protection from physical trauma. For example, the backing layer 120 may be constructed from a material that can reduce evaporative losses and provide a fluid seal between two components or two environments, such as between a therapeutic environment and a local external environment. The backing layer 120 may be formed from a suitable material, such as a polymer, for example, which may comprise or be an elastomeric film or membrane that can provide a seal at a, tissue site. In some embodiments, the backing layer 120 may comprise or be a polyurethane. In some embodiments, the backing layer 120 may have a high moisture-vapor transmission rate (MVTR). For example, in such an embodiment, the MVTR may be at least 300 g/m² per twenty-four hours. For example, the backing layer 120 may comprise a polymer drape, such as a polyurethane film, that may be permeable to water vapor but generally impermeable to liquid water. In some embodiments, the backing or drape may have a thickness in the range of about from about 15 to about 50 microns.

### Wound Dressing—Secondary Layer

Additionally, in some embodiments, the wound dressing 100 may further comprise a secondary layer, for example, positioned between the dressing layer 110 and the backing layer 120. In some embodiments, the secondary layer may comprise fluid pathways interconnected so as to improve distribution or collection of fluids. For example, in some embodiments, the secondary layer may be a porous material having a plurality of interconnected cells or pores. Suitable examples of porous material include a cellular foam such as an open-cell foam, a reticulated foam, or porous tissue collections. Other suitable porous material may include gauze or felted mat, which generally include pores, edges, or walls adapted to form interconnected fluid pathways. For example, in some embodiments, the secondary layer may be a foam having pore sizes in a range of 400-600 microns. In one non-limiting example, the secondary layer may be reticulated polyurethane foam.

In some embodiments having a secondary layer, the secondary layer may be characterized as exhibiting absorbency. For example, the secondary layer may exhibit an absorbency of at least 3 g saline/g, particularly at least 5 g saline/g, from 5 to 50 g saline/g, from 8 to 40 g saline/g, or from 8 to 20 g saline/g. In some embodiments, the secondary layer may be hydrophilic. In an example in which the secondary layer may be hydrophilic, the secondary layer may also wick fluid away from a dressing layer 110. In such embodiments, the wicking properties of the secondary layer may draw fluid away from dressing layer 110 by capillary flow or other wicking mechanisms. An example of a hydrophilic foam is a polyvinyl alcohol, open-cell foam. Other hydrophilic foams may include those made from or containing a polyether or a polyurethane. Additional or alternative foams that may exhibit hydrophilic characteristics include hydrophobic foams that have been treated or coated to provide hydrophilicity.

### Other Optional Dressing Layers

In some embodiments, the dressing layer 110 may be in contact with and adhered to the backing layer 120. This adherence may, in some embodiments, result from an adherent layer disposed between the dressing layer 110 and the backing layer 120, thus constituting direct adherence. Such direct adherence means that the adherent layer, or at least the portion disposed between the dressing layer 110 and the backing layer 120, can be comprised of one or more different kinds of physical or chemical adhesive compositions. The adherent layer or portion thereof, in some embodiments, would not be expected to directly contact a tissue site. In embodiment's with a backing layer margin extending beyond the dressing layer 110, the adherent layer may typically extend out to cover all or part of the backing layer margin. In such embodiments, the portion of the adherent layer on the margin may adhere the wound dressing 100 to a tissue site. Adherents that may directly contact tissue or that may be exposed to a treatment environment can typically have additional requirements, such as biocompatibility, and may be selected from a smaller list of physical or chemical adhesive compositions.

In some embodiments, such as where an adherent layer is an external layer in the wound dressing 100, for example to adhere the wound dressing to a tissue site, the adherent layer can be releasably coupled to a release liner configured for removal before application to a tissue site, for example.

Adherence between the dressing layer 110 and the backing layer 120 may additionally or alternatively be indirect. For example, in some embodiments with a backing layer margin extending beyond the dressing layer 110, the adherent layer may be disposed on the backing layer margin and extend further over some portion of the dressing layer 110, such as the margin of the dressing layer 110. If this occurs without an adherent layer between the backing layer 120 and the dressing layer 110, the adherent layer may be said to indirectly adhere those layers, because those layers are each adhered to the adherent layer but not directly to each other. Such a configuration can allow an absorbent portion of the dressing layer 110 to expand differentially from the backing layer 120, for instance enabling relatively high levels of absorption of fluid with additional degrees of freedom. If the backing layer 120 is directly adhered to the dressing layer 110, the absorbent portion of the dressing layer 110 may expand into the tissue site, which can in some embodiments create undesirable pressure on a tissue site.

In particular embodiments, the adherent layer may comprise a hydrocolloid material.

In some embodiments, absorbent material may be absent in or removed from a zone within the absorption layer. Such embodiments offer an additional or alternative mechanism enabling at least partial fluid absorptive expansion within the absorption layer, which can enable additional degrees of freedom for fluid absorption while creating no or little additional pressure on the wound site. For example, by creating a central zone in the absorbent layer of dressing layer 110 that is absent of material, the other portions of the absorbent layer can have extra volume to expand and can optionally experience increased fluid flow within the dressing layer 110, thus rendering the absorbent layer more efficient.

In some embodiments, the absorbent layer may be perforated to increase fluid flow, to reduce time to equilibrium absorption, or both. Such embodiments offer another additional or alternative mechanism enabling additional degrees of freedom for fluid absorption while creating no or little additional pressure on the wound site.

In some embodiments, the wound dressing 100 may include a contact layer, which may be non-adherent. For example, a contact layer may be disposed over the second surface of the dressing layer 110, opposite the backing layer 120. Non-adherent contact layers may be particularly advantageous in fibrinous situations to reduce potential adherence of the dressing layer 110 to a tissue site, to enable fluid to be effectively drawn away from the tissue site through the contact layer, or both. In some embodiments, therefore, the contact layer may be perforated, for example, for increased fluid flow. In various embodiments, the contact layer may comprise at least one of: an alkyl acrylate polymer, such as a methyl acrylate polymer, an ethyl acrylate polymer, or the like; an alkacrylate polymer, such as a methacrylate polymer, an ethacrylate polymer, or the like; and an alkyl alkacrylate polymer, such as a methyl methacrylate polymer, an ethyl methacrylate polymer, a methyl ethacrylate polymer, an ethyl ethacrylate polymer, or the like. Such (alk)acrylate polymers may be homopolymers but are more often copolymers, for example, with olefin comonomers. In particular, the non-adherent layer may comprise an ethylene-methyl acrylate copolymer, such as used in TIELLE™ Dressings and in STLVERCEL™ NON-ADHERENT Dressings available from Systagenix Wound Management, Limited. In various embodiments, the contact layer may comprise a silicone or polysiloxane polymer or copolymer. In such embodiments, the contact layer can extend over the absorbent layer, over the composite island, or over the dressing layer, as applicable, and over at least a portion of the backing layer margin. By extending over at least a portion of the backing layer margin, the portion of the contact layer extending over the backing layer margin may be coupled to the backing layer via the adherent layer. Optionally, the contact layer may have margin perforations, for example through which access can be allowed for the adherent layer to contact a wound site or tissue proximal thereto. Additionally or alternatively, particularly if the contact layer does not have sufficient tack to adhere the wound dressing to a wound site or to tissue proximal thereto, a second adherent layer may optionally be disposed on a surface of the contact layer opposite the backing layer, absorbent layer, composite island, or dressing layer, if any or all are present.

### Negative-Pressure Therapy

Additionally, in some embodiments, a dressing layer such as dressing layer 110, or a dressing comprising such a dressing layer, such as the wound dressing 100, may be employed in therapy, for example to treat a tissue site with reduced pressure. Treatment with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, micro-deformation of tissue at a wound site, and combinations thereof. Individually or together, these benefits may increase development of granulation tissue and reduce healing times.

In various embodiments, a negative-pressure therapy system may generally include a negative-pressure supply, and may include or be configured to be coupled to a distribution component. In general, a distribution component may refer to any complementary or ancillary component configured to be fluidly coupled to a negative-pressure supply in a fluid path between a negative-pressure supply and a tissue site.

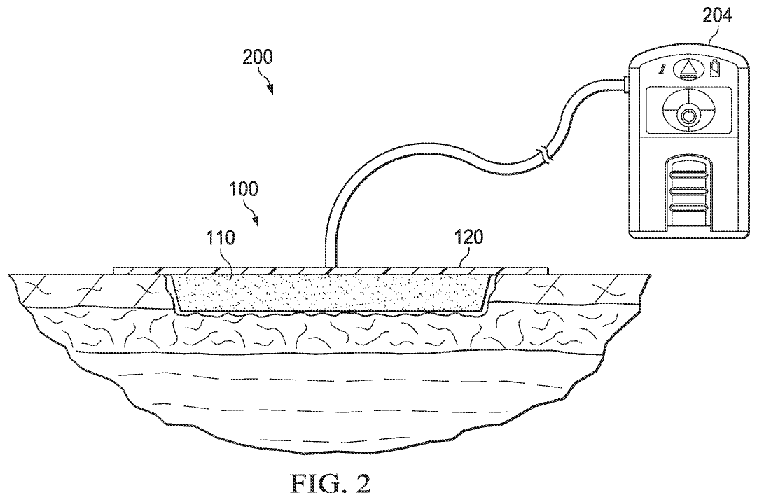

FIG. 2 is a simplified schematic of an example embodiment of a therapy system 200 that can provide negative-pressure therapy to a tissue site. In the example embodiment of FIG. 2, the wound dressing 100 is fluidly coupled to a negative-pressure source 204, such that negative pressure may be applied to a tissue site via the wound dressing 100.

For example, the dressing layer 110 may be generally configured to distribute negative pressure across a tissue site. For example, in some embodiments, the dressing layer 110 may comprise or be configured as a manifold. A “manifold” in this context generally includes any composition or structure providing a plurality of pathways configured to collect or distribute fluid across a tissue site under pressure. For example, a manifold may be configured to receive negative pressure from a negative-pressure source and to distribute negative pressure through multiple apertures or pores, which may have the effect of collecting fluid and drawing the fluid toward the negative-pressure source. More particularly, in the embodiment of FIG. 2, the dressing layer 110 is configured to receive negative pressure from the negative-pressure source 204 and to distribute negative pressure across the dressing layer 110. For example, this may have the effect of collecting fluid from a sealed space, such as by drawing fluid from the tissue site through the dressing layer 110. In additional or alternative embodiments, the fluid path(s) may be reversed or a secondary fluid path may be provided to facilitate movement of fluid across a tissue site. In some embodiments, the fluid pathways of a manifold may be interconnected to improve distribution or collection of fluids. In some embodiments, a manifold may comprise or be a porous foam material having a plurality of interconnected cells or pores. For example, open-cell foams generally include pores, edges, walls, or combinations thereof that may form interconnected fluid pathways, such as channels.

The fluid mechanics associated with using a negative-pressure source to reduce pressure in another component or location, such as within a sealed therapeutic environment, may be mathematically complex. However, the basic principles of fluid mechanics applicable to negative-pressure therapy are generally well-known to those skilled in the art. The process of reducing pressure may be described generally and illustratively herein as “delivering,” “distributing,” or “generating” negative pressure, for example.

In general, a fluid such as exudate flows toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. This orientation is generally presumed for purposes of describing various features and components herein. However, the fluid path may also be reversed in some applications, such as by substituting a positive-pressure source for a negative-pressure source, and this descriptive convention should not be construed as a limiting convention.

As used herein, “negative pressure” is generally intended to refer to a pressure less than a local ambient pressure, such as the ambient pressure in a local environment external to a sealed therapeutic environment provided by the wound dressing 100. In many cases, the local ambient pressure may also be the atmospheric pressure proximate to or about a tissue site. Additionally or alternatively, the pressure may be less than a hydrostatic pressure associated with the tissue at the tissue site. Unless otherwise indicated, values of pressure stated herein are gauge pressures. Similarly, references to increases in negative pressure typically refer to a decrease in absolute pressure, for example a more negative pressure, while decreases in negative pressure typically refer to an increase in absolute pressure, for example a less negative pressure or a more positive pressure. While the amount and nature of negative pressure applied to a tissue site may vary according to therapeutic requirements, the pressure is generally a low vacuum, also commonly referred to as a rough vacuum, for example between −5 mm Hg (−667 Pa) and −500 mm Hg (−66.7 kPa). Common therapeutic ranges can be between −75 mm Hg (−9.9 kPa) and −300 mm Hg (−39.9 kPa).

In various embodiments, a negative-pressure supply, such as the negative-pressure source 204, may be a reservoir of air at a negative pressure. Alternatively, negative-pressure source 204 may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components that further facilitate therapy, such as sensors, processing units, alarm indicators, memory, databases, software, display devices, or user interfaces. For example, in some embodiments, the negative-pressure source 204 may be combined with a controller and other components into a therapy unit. A negative-pressure supply may have one or more supply ports configured to facilitate coupling and de-coupling of the negative-pressure supply to one or more distribution components.

In various embodiments, components may be fluidly coupled to each other to provide a path for transferring fluids between the components. For example, components may be fluidly coupled through a fluid conductor, such as a tube. As used herein, the term “fluid conductor” is intended to broadly include a tube, pipe, hose, conduit, or other structure with one or more lumina adapted to convey a fluid between two ends thereof. Typically, a fluid conductor may be an elongated, cylindrical structure with some flexibility, but the geometry and rigidity may vary. In some embodiments, the negative-pressure source 204 may be operatively coupled to the wound dressing 100 via a dressing interface. For example, in the embodiment of FIG. 2, the wound dressing 100 may be coupled to the negative-pressure source 204 via a dressing interface to receive negative pressure.

### Methods of Use

Also disclosed herein are methods of treating a tissue site, for example, in the context of various therapies, such as eliminating, minimizing, or reducing edema, such as post-operative edema, particularly in areas of relatively high articulation or flexure. Non-limiting examples of areas implicating relatively high articulation or flexure include shoulder, elbow, knee, ankle, or hip joints, particularly knee or elbow joints.

In some embodiments, a therapy method may comprise positioning the dressing layer 110 with respect to a tissue site. For example, in operation, the dressing layer 110 may be positioned proximate to the tissue site. For example, the dressing layer 110 may be placed within, over, on, or otherwise proximate to the tissue site. The dressing layer 110 may be used with any of a variety of wounds, such as those occurring from trauma, surgery, or disease. Additionally, in some embodiments, a cover such as the backing layer 120 may be placed over the dressing layer 110 and sealed to an attachment surface near the tissue site. For example, the backing layer 120 may be sealed to undamaged epidermis peripheral to a tissue site. In some embodiments, the dressing layer 110 may be positioned and the backing layer 120 may be positioned thereafter. In some embodiments, the dressing layer 110 and backing layer 120 may be preassembled, for example, such that the dressing layer 110 and backing layer 120 are positioned with respect to each other prior to placement proximate the tissue site. Thus, the backing layer 120 can provide a sealed therapeutic environment including the dressing layer 110 and proximate to a tissue site, substantially isolated from the external environment.

Additionally, in some negative-pressure therapy embodiments, a negative-pressure therapy may comprise positioning the dressing layer 110 and backing layer 120 proximate to a tissue site. For example, the various components of the dressing layer 110 may be positioned with respect to the tissue site sequentially or, alternatively, may be positioned with respect to each other and then positioned with respect to the tissue site. The negative-pressure therapy may further comprise sealing the dressing layer 110 to tissue surrounding the tissue site to form a sealed space. For example, the backing layer 120 may be placed over the dressing layer 110 and sealed to an attachment surface near the tissue site, such as undamaged epidermis peripheral to a tissue site. Thus, the dressing layer 110 and backing layer 120 can provide a sealed therapeutic environment proximate to the tissue site, substantially isolated from the external environment.

The negative-pressure therapy method may further comprise fluidly coupling a negative-pressure source to the sealed space and operating the negative-pressure source to generate a negative pressure in the sealed space. For example, the negative-pressure source 204 may be coupled to the wound dressing 100 such that the negative-pressure source 204 may be used to reduce the pressure in the sealed space. For example, negative pressure applied across the tissue site via the wound dressing 100 may be effective to induce macrostrain and microstrain at the tissue site, as well as to remove exudates and other fluids from the tissue site.

### EMBODIMENTS

Additional or alternative examples may include one or more of the following embodiments.

### Embodiment 1

A wound dressing comprising: a composite island configured to collectively exhibit substantially elastic recovery under wound treatment conditions and comprising a foam layer and an absorbent layer coupled to the foam layer, the foam layer exhibiting substantially elastic recovery under wound treatment conditions and the absorbent layer not exhibiting substantially elastic recovery under wound treatment conditions; a backing layer having a margin that extends beyond the composite island; and an adherent layer disposed on at least the margin of the backing layer.

### Embodiment 2

A wound dressing comprising: a perforated envelope configured to exhibit substantially elastic recovery under wound treatment conditions; an absorbent layer surrounded by the perforated envelope and having a plurality of fenestrations configured to mitigate plastic deformation of the absorbent layer; a backing layer having a margin that extends beyond the perforated envelope; and an adherent layer disposed on at least the margin of the backing layer.

### Embodiment 3

A wound dressing comprising: a composite island configured to collectively exhibit substantially elastic recovery under wound treatment conditions and comprising a foam layer and an absorbent layer coupled to the foam layer, the foam layer exhibiting substantially elastic recovery under wound treatment conditions and the absorbent layer having a plurality of fenestrations collectively configured to mitigate plastic deformation of the absorbent layer and not exhibiting substantially elastic recovery under wound treatment conditions; a backing layer having a margin that extends beyond the composite island; and an adherent layer disposed on at least a margin of the backing layer and extending over a margin of the composite island, thereby fastening the adherent layer to the margin of the composite island.

### Embodiment 4

A wound dressing comprising: an absorbent layer having a plurality of fenestrations configured to mitigate plastic deformation of the absorbent layer; a backing layer having a margin that extends beyond the absorbent layer; and an adherent layer disposed between the absorbent layer and the backing layer and extending onto the margin of the backing layer, thereby forming an adherent layer margin, wherein each of the plurality of fenestrations extend inward from an edge of the absorbent layer no further than a midpoint between opposing edges, so as to form a central backbone with appendages extending outward therefrom.

### Embodiment 5

A wound dressing comprising: an absorbent layer having a plurality of fenestrations configured to mitigate plastic deformation of the absorbent layer; a backing layer having a margin that extends beyond the absorbent layer; and an adherent layer disposed between the absorbent layer and the backing layer and extending onto the margin of the backing layer, thereby forming an adherent layer margin, wherein at least a majority of the plurality of fenestrations are oriented to form an acute angle between each fenestration and a width or length direction.

### Embodiment 6

The wound dressing of any of embodiments 1-5, wherein the adherent layer comprises a hydrocolloid.

### Embodiment 7

The wound dressing of any of embodiments 1-6, wherein the absorbent layer comprises: (i) from about 45% to about 90% of cellulose ether fibers; and (ii) from about 10% to about 55% of reinforcing fibers.

### Embodiment 8

The wound dressing of embodiment 7, wherein the cellulose ether fibers are composed of at least one of carboxymethyl cellulose, carboxylethyl cellulose, hydroxyethyl cellulose, hydroxypropyl cellulose, and hydroxypropylmethyl cellulose.

### Embodiment 9

The wound dressing of embodiment 7 or embodiment 8, wherein the reinforcing fibers are composed of at least one of a polyurethane gel, an amide polymer, an olefin polymer, an ester polymer, and a modified acrylamide polymer.

### Embodiment 10

The wound dressing of any of claims 1, 4, and 6-9, wherein the absorbent layer has a plurality of fenestrations collectively configured to mitigate plastic deformation of the absorbent layer.

### Embodiment 11

The wound dressing of any of embodiments 1, 3, and 6-10, wherein the foam layer is coupled to the absorbent layer by lamination.

### Embodiment 12

The wound dressing of any of embodiments 1, 3, and 6-11, wherein a surface of the absorbent layer not coupled to the foam layer is tissue-facing.

### Embodiment 13

The wound dressing of any of embodiments 1, 3, and 6-11, wherein a surface of the foam layer not coupled to the absorbent layer is tissue-facing.

### Embodiment 14

The wound dressing of any of embodiments 1, 3, 6-11, and 13, wherein the absorbent layer coupled to the foam layer is absent in at least a portion of a central zone of its length, such that a surface of the foam layer not coupled to the absorbent layer is tissue-facing.

### Embodiment 15

The wound dressing of any of embodiments 1-14, further comprising a non-adherent layer.

### Embodiment 16

The wound dressing of embodiment 15, wherein the non-adherent layer is perforated and comprises at least one of an alkyl acrylate polymer, an alkacrylate polymer, and an alkyl alkacrylate polymer.

### Embodiment 17

The wound dressing of embodiment 16, wherein the alkyl acrylate polymer comprises an ethylene-methyl acrylate copolymer.

### Embodiment 18

The wound dressing of any of embodiments 2-4 and 6-17, wherein the wound dressing has a width and a length perpendicular to the width, and wherein at least a majority of the plurality of fenestrations are oriented parallel to the width.

### Embodiment 19

The wound dressing of any of embodiments 1-18, wherein the absorbent layer further has a plurality of perforations.

### Embodiment 20

The wound dressing of any of embodiments 1-19, wherein the backing layer is non-adherent and comprises a polyurethane.

### Embodiment 21

A method of eliminating, minimizing, or reducing edema for a wound surrounded by tissue, the method comprising positioning a wound dressing according to any of claims 1-20 over the wound, such that at least a portion of the adherent layer contacts the tissue.

### Embodiment 22

The method of embodiment 21, wherein the wound is proximal to a knee joint or an elbow joint.

### Embodiment 23

The method of embodiment 21 or embodiment 22, further comprising: sealing the wound dressing within a sealed space near the wound; and applying a negative pressure to the sealed space.

### EXAMPLES

One, some, or all of the advantages associated with the disclosed compositions, wound dressings, methods of making, and methods of using or treating may be further demonstrated by the following non-limiting examples.

Elements of each exemplary embodiment are generally labelled to have their last two digits in common with similar elements in other exemplary embodiments. For instance, wound dressings herein are labelled to end in 00; dressing layers are labelled to end in 10; backing layers are labelled to end in 20; etc. The difference in the other digit(s) is(are) indicative of alternative exemplary embodiments.

### Example 1

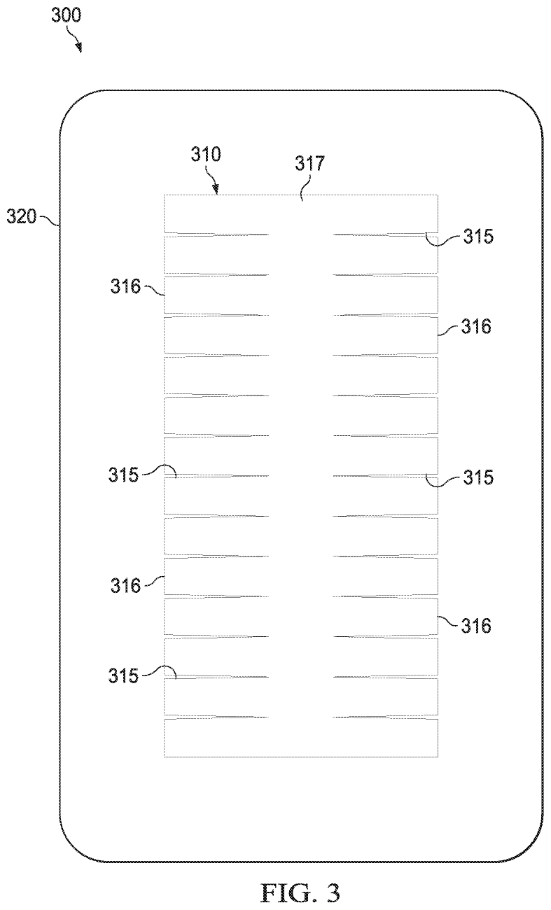

FIG. 3 shows an example of a backbone-style wound dressing 300. In FIG. 3, the wound dressing 300 includes a backing layer 320 and a dressing layer 310. In this Example, the backing layer 320 may be a polyurethane backing layer, and the dressing layer 310 can be a CMC-based non-woven absorbent layer having a plurality of through-thickness fenestrations 315 extending inward from the edges almost but not all the way to the midpoint of the width. The plurality of fenestrations 315 are configured to give the dressing layer 310 a backbone 317, outward from which a plurality of appendages 316 extend. In the example of FIG. 3, an adherent layer 330 is coupled to both the backing layer 320 and the dressing layer 310. The absorbent layer 310 may be smaller than both the backing layer 320 and the adherent layer 330, so as to define a margin (not labelled). The adherent layer 330 on this margin can be operable to removably adhere the wound dressing 300 to tissue, such as at a wound site, and can advantageously be an acceptable medical tissue adhesive, such as a hydrocolloid. The dressing layer 310 typically does not exhibit substantially elastic recovery with the application of a strain and has fenestrations 315, each with a slit length approximately perpendicular to the likely strain direction. Nevertheless, the fenestrations 315 can be configured to collectively mitigate plastic deformation of the dressing layer 310 via its coupling to the adherent layer 330. The application of a strain is meant to simulate or to approximate the duty of wound dressing 300 when positioned over wound sites in areas of relatively high flexure (particularly knee and/or elbow joints), for example, thereby eliminating, minimizing, or reducing edema at such wound sites.

### Example 2

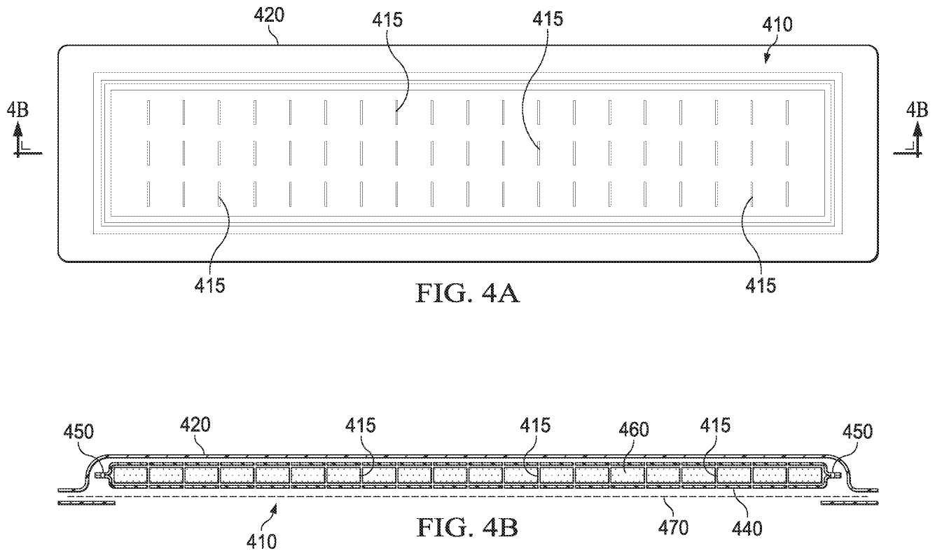

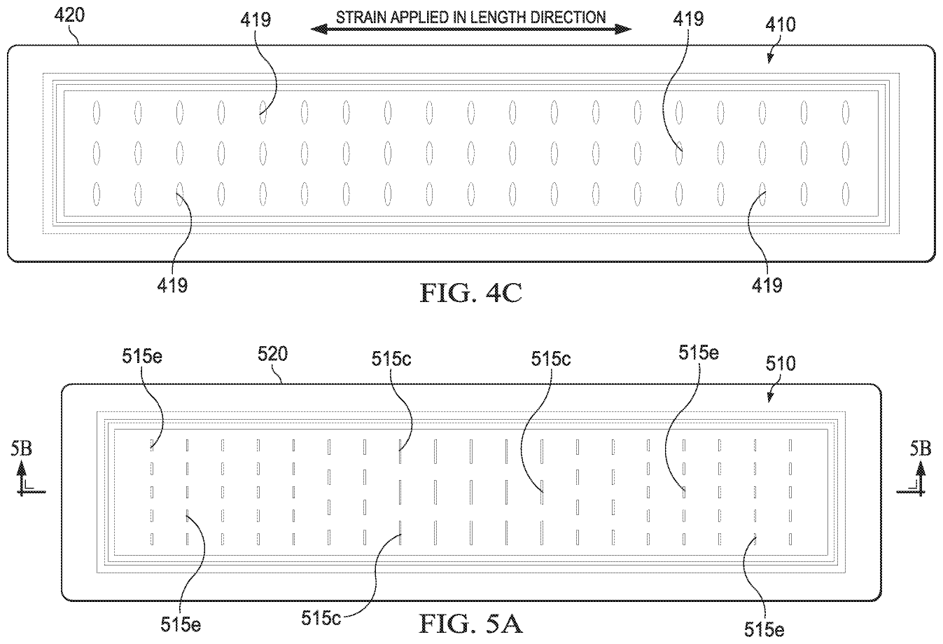

FIGS. 4A-4C show various views of an exemplary wound dressing 400 with an enveloped composite dressing layer 410. FIG. 4A shows a top view of the wound dressing 400, which also identifies a polyurethane backing layer 420. As can be seen in side view, in FIG. 4B, the dressing layer 410 is comprised of a central CMC-based non-woven absorbent layer 460 having a plurality of through-thickness fenestrations 415, surrounded by a polyurethane envelope 440 sealed by edge welds 450. In this Example, the polyurethane envelope 440 is perforated (not labelled) to allow fluid to access the absorbent layer 460 contained therein. The envelopment of the absorbent layer 460 within the polyurethane envelope 440 achieves an indirect linkage between the two layers, without direct coupling. The fenestrated CMC-based non-woven absorbent layer 460 typically does not exhibit substantially elastic recovery with the application of a strain, and the polyurethane envelope 440 typically does exhibit substantially elastic recovery, for example, by virtue of its edge welds 450. In this Example, the combination of the polyurethane envelope 440 and the fenestrations 415 are configured to impart substantial elastic recovery to the absorbent layer 460, thus imbuing the dressing layer 410 with substantial elastic recovery with the application of a strain in a direction approximately perpendicular to the fenestration slit length.

The application of a strain is meant to simulate or to approximate the duty of wound dressing 400 when positioned over wound sites in areas of relatively high flexure (particularly knee or elbow joints), for example, thereby eliminating, minimizing, or reducing edema at such wound sites, as shown in FIG. 4C. FIG. 4C shows the strain-expanded fenestrations 419, for example, that can be configured to substantially recover to their original shape). Additionally, the wound dressing 400 may include a non-adherent layer 470, which can enable fluid to be effectively drawn away from the wound site through the non-adherent layer 470. Optionally, the non-adherent wound contacting layer may be perforated (not shown). Also not shown but typically present is an adherent layer on at least a margin of the backing layer 420 to allow removable adherence to tissue, such as proximal to a wound site.

### Example 3

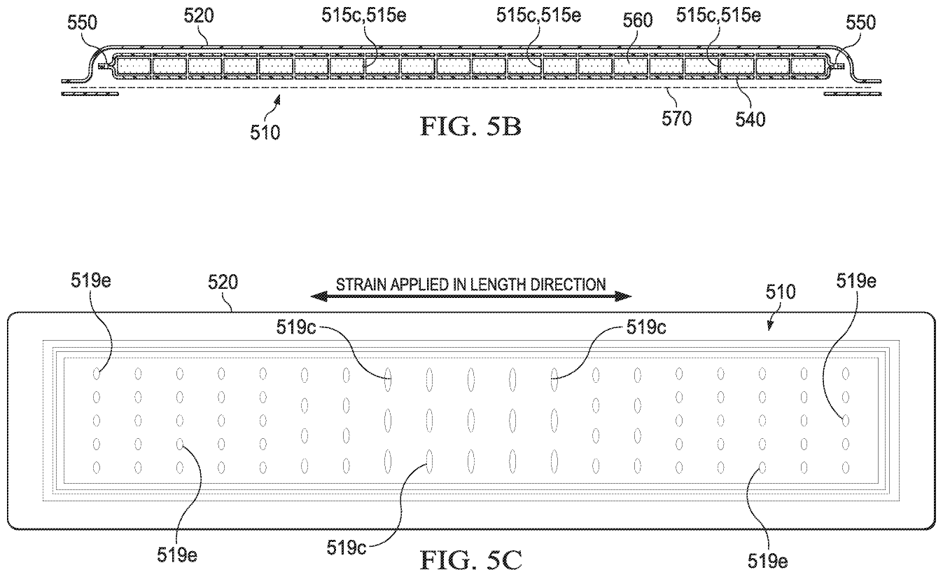

FIGS. 5A-5C show various views of an exemplary wound dressing 500 with an enveloped composite dressing layer 510. FIG. 5A shows a top view of wound dressing 500, which also identifies a polyurethane backing layer 520. As can be seen in side view, in FIG. 5B, dressing layer 510 is comprised of a central CMC-based non-woven absorbent layer 560, surrounded by a polyurethane envelope 540 sealed by edge welds 550. In this Example, different from Example 2, there are two different sizes of fenestrations in the absorbent layer 560—fenestrations in the central region of the layer are larger fenestrations 515c and in the bounding edge regions of the layer are smaller fenestrations 515e. In this Example, the polyurethane envelope 540 is perforated (not labelled) to allow fluid to access the absorbent layer 560 contained therein. The envelopment of the absorbent layer 560 within the polyurethane envelope 540 achieves an indirect linkage between the two layers, without direct coupling. The fenestrated (515c, 515e) CMC-based non-woven absorbent layer 560 typically does not exhibit substantially elastic recovery with the application of a strain, and the polyurethane envelope 540 typically does exhibit substantially elastic recovery, for example, by virtue of its edge welds 550. In this Example, the combination of the polyurethane envelope 540 and the fenestrations 515c, 515e are configured to impart substantial elastic recovery to the absorbent layer 560, thus imbuing the dressing layer 510 with substantial elastic recovery with the application of a strain in a direction approximately perpendicular to the fenestration slit length.

The application of a strain is meant to simulate or to approximate the duty of wound dressing 500 when positioned over wound sites in areas of relatively high flexure (particularly knee or elbow joints), for example, thereby eliminating, minimizing, or reducing edema at such wound sites, as shown in FIG. 5C. FIG. 5C shows the strain-expanded fenestrations 519c, 519e, for example, that can be configured to substantially recover to their original slit-shape (515c, 515e). The smaller fenestrations 515e may optionally be absent in some embodiments, leaving only fenestrations 515c in the central region of the layer. Without being bound by theory, it is believed that the application of strain need not be distributed across the entire absorbent layer 560 or dressing layer 510 equally, but may instead be distributed predominantly over only a portion of the layer(s), such as in the central region that is most likely to be positioned proximal to the wound site. Additionally, the wound dressing 500 may include a non-adherent layer 570, which can enable fluid to be effectively drawn away from the wound site through the non-adherent layer 570. Optionally, the non-adherent layer can be wound contacting and perforated (not shown). Also not shown but typically present is an adherent layer on at least a margin of the backing layer 520 to allow removable adherence to tissue, such as proximal to a wound site.

### Example 4

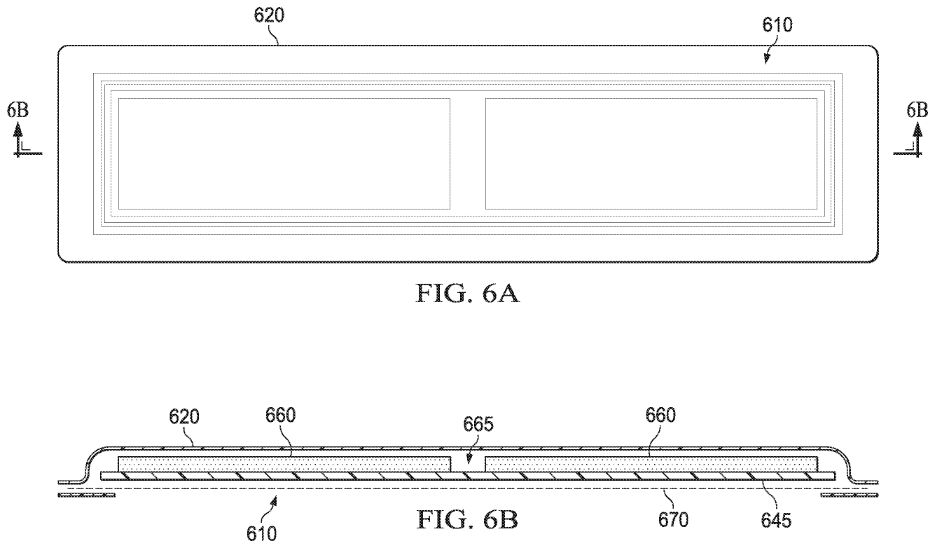

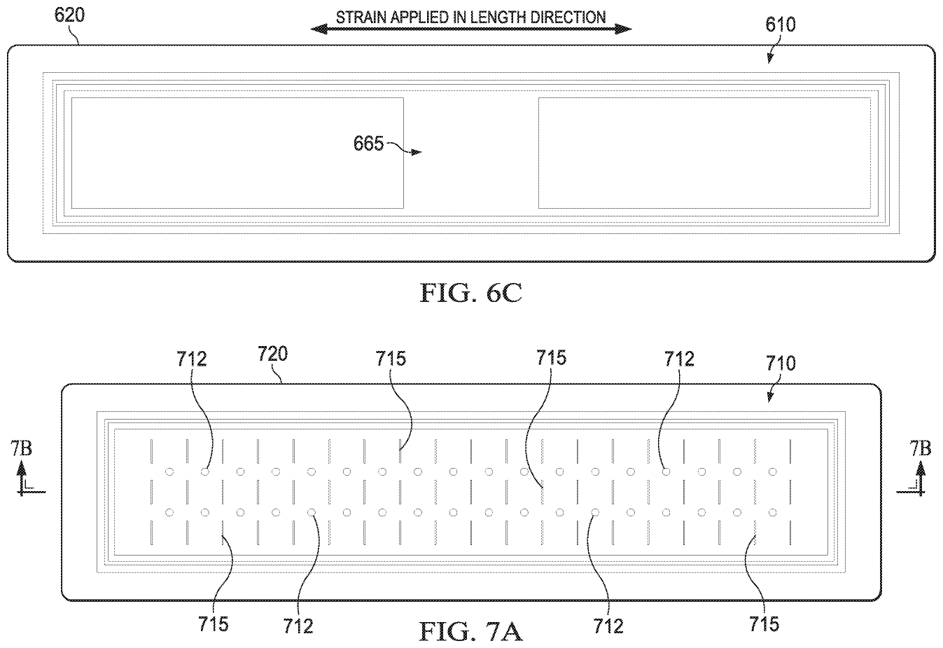

FIGS. 6A-6C show various views of an exemplary wound dressing 600 with an enveloped composite dressing layer 610. FIG. 6A shows a top view of wound dressing 600, which also identifies a polyurethane backing layer 620. As can be seen in side view, in FIG. 6B, dressing layer 610 is a multi-layer composite comprised of a CMC-based non-woven absorbent layer 660 coupled (in this case, laminated) to an open cell polyurethane foam layer 645. In this Example, the absorbent layer 660 is absent in at least a portion 665 of a central zone of its length. This absence can allow for at least partial fluid absorptive expansion within the absorption layer 660, and can potentially allow increased fluid flow within the dressing layer 610 from the extra volume available. Also in this Example, the foam layer 645 typically exhibits substantially elastic recovery with the application of a strain, and the absorbent layer 660 typically does not exhibit substantially elastic recovery with the application of a strain. By virtue of the coupling of these two layers, the composite dressing layer 610 can exhibit substantially elastic recovery at wound treatment conditions. Optionally, the absorption layer 660 may be through-thickness fenestrated (not shown), if desired.

The application of a strain is meant to simulate or to approximate the duty of wound dressing 600 when positioned over wound sites in areas of relatively high flexure (particularly knee or elbow joints), for example, thereby eliminating, minimizing, or reducing edema at such wound sites, as shown in FIG. 6C. Additionally, the wound dressing 600 may include a non-adherent layer 670, which can enable fluid to be effectively drawn away from the wound site through the non-adherent layer 670. Optionally, the non-adherent layer may be wound contacting and perforated (not shown). Though this Example shows the foam layer 645 closer to the wound site surface than the absorbent layer 660, the opposite configuration is also possible and envisioned. Also not shown but typically present is an adherent layer on at least a margin of the backing layer 620 to allow removable adherence to tissue, such as proximal to a wound site.

### Example 5

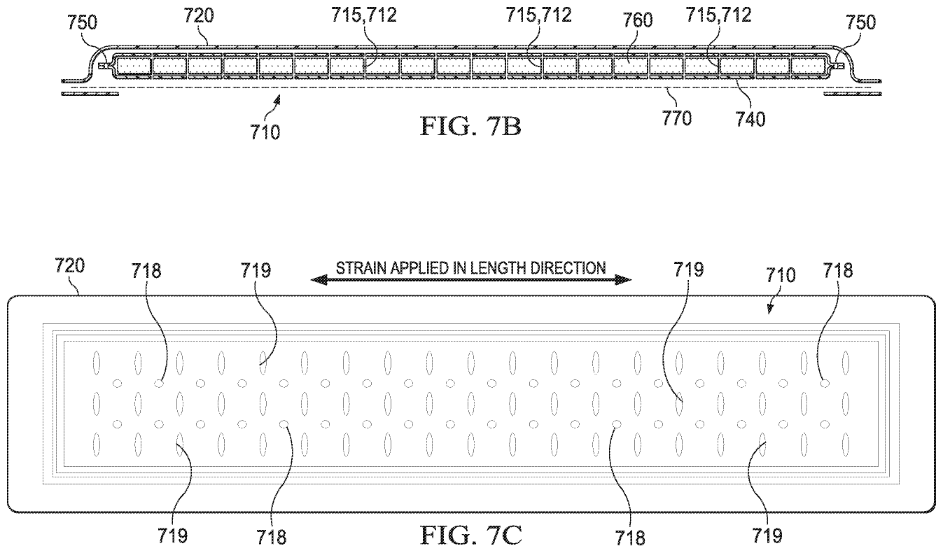

This Example is similar to Example 2 (FIGS. 4A-4C), but with the presence of perforations in the absorbent layer, in addition to fenestrations. FIGS. 7A-7C show various views of an exemplary wound dressing 700 with an enveloped composite dressing layer 710. FIG. 7A shows a top view of wound dressing 700, which also identifies a polyurethane backing layer 720. As can be seen in side view, in FIG. 7B, dressing layer 710 is comprised of a central CMC-based non-woven absorbent layer 760 having a plurality of through-thickness fenestrations 715 and a plurality of perforations 712, surrounded by a polyurethane envelope 740 sealed by edge welds 750. In this Example, the polyurethane envelope 740 is perforated (not labelled) to allow fluid to access the absorbent layer 760 contained therein. The envelopment of the absorbent layer 760 within the polyurethane envelope 740 achieves an indirect linkage between the two layers, without direct coupling. The fenestrated (715) and perforated (712) CMC-based non-woven absorbent layer 760 typically does not exhibit substantially elastic recovery with the application of a strain, and the polyurethane envelope 740 typically does exhibit substantially elastic recovery, for example, by virtue of its edge welds 750. In this Example, the combination of the polyurethane envelope 740, the fenestrations 715, and the perforations 712 are configured to impart substantial elastic recovery to the absorbent layer 760, thus imbuing the dressing layer 710 with substantial elastic recovery with the application of a strain in a direction approximately perpendicular to the fenestration slit length.

The application of a strain is meant to simulate or to approximate the duty of wound dressing 700 when positioned over wound sites in areas of relatively high flexure (particularly knee or elbow joints), for example, thereby eliminating, minimizing, or reducing edema at such wound sites, as shown in FIG. 7C. FIG. 7C shows the strain-expanded fenestrations 719 and strain-expanded perforations 718, for example, that can be configured to substantially recover to their original shapes (715, 712). Additionally, the wound dressing 700 can include a non-adherent layer 770, which can enable fluid to be effectively drawn away from the wound site through the non-adherent layer 770. Optionally, the non-adherent layer can be wound contacting and perforated (not shown). Also not shown but typically present is an adherent layer on at least a margin of the backing layer 720 to allow removable adherence to tissue, such as proximal to a wound site.

### Example 6

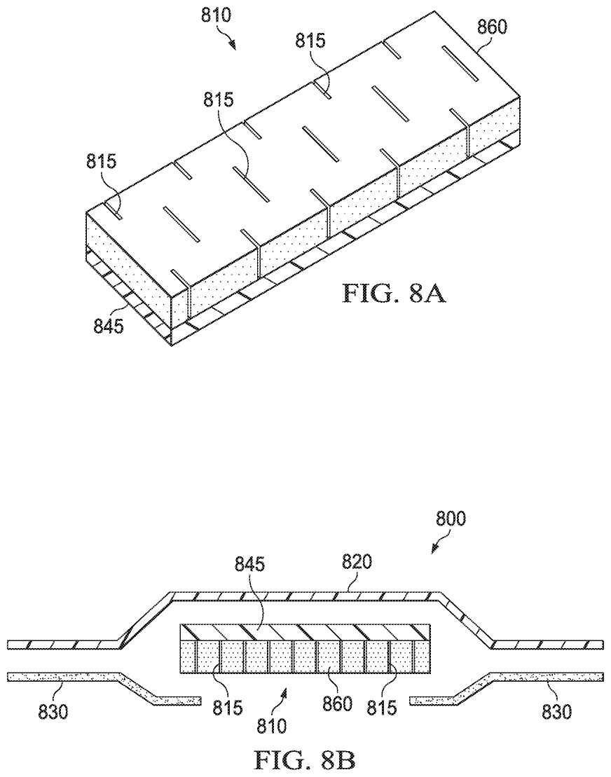

FIG. 8A shows an exemplary dual-layer composite dressing layer 810. The dressing layer 810 is comprised of a CMC-based non-woven absorbent layer 860 having a plurality of through-thickness fenestrations 815 and coupled (in this case, laminated) to an open cell polyurethane foam layer 845. In this Example, the foam layer 845 typically exhibits substantially elastic recovery with the application of a strain, and the fenestrated (815) absorbent layer 860 typically does not exhibit substantially elastic recovery with the application of a strain. Also in this Example, the combination of the coupling of these two layers and the fenestrations 815 are configured to impart substantial elastic recovery to the absorbent layer 860, thus imbuing the dressing layer 810 with substantial elastic recovery with the application of a strain in a direction approximately perpendicular to the fenestration slit length. Such an applied strain is meant to simulate or to approximate the duty of the wound dressing 800, shown in FIG. 8B, when positioned over wound sites in areas of relatively high flexure (particularly knee or elbow joints), for example, thereby eliminating, minimizing, or reducing edema at such wound sites. Though this Example shows the foam layer 845 closer to the wound site surface than the absorbent layer 860, the opposite configuration is also possible and envisioned.

FIG. 8B shows a side view of a wound dressing 800 comprising the composite dressing layer 810 of FIG. 8A. In FIG. 8B, a polyurethane backing layer 820 is shown over the dressing layer 810. In this Example, the adherent layer 830 is coupled to a margin of the backing layer 820 to allow removable adherence to tissue, such as proximal to a wound site. In this Example, the adherent layer 830 also extends to cover and is coupled directly to a margin of composite dressing layer 810 as well. By virtue of their coupling through the adherent layer 830, the backing layer 820 and the dressing layer 810 layers are only indirectly coupled to each other. This configuration can allow the absorbent layer 860 to expand differentially from the backing layer 820, thereby enabling relatively high levels of fluid absorption with additional degrees of freedom. Thus, though the dressing layer 810 may additionally be adhered directly to the backing layer 820, the indirect coupling configuration is believed to have one or more advantages.

### Example 7

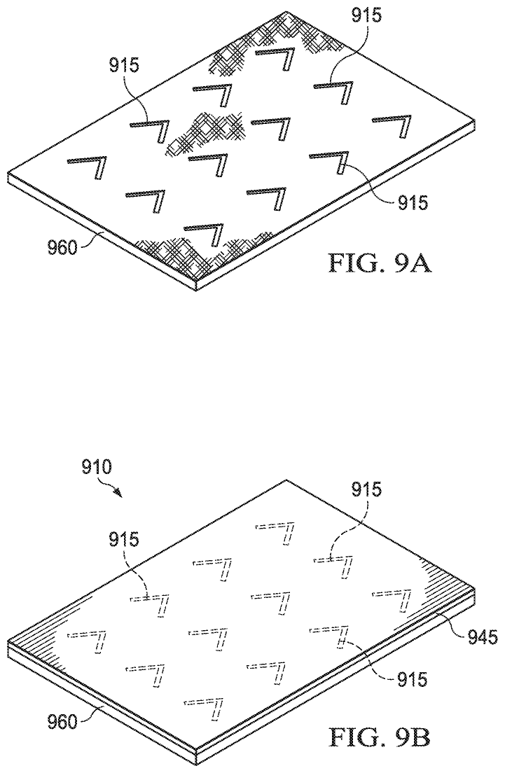

FIG. 9A shows an exemplary absorbent layer 960 comprised of a CMC-based non-woven material having a plurality of through-thickness fenestrations 915. In this Example, a majority of the fenestrations 915 are oriented to form an acute angle between each fenestration and a width or length of the absorbent layer 960. In FIG. 9A, these fenestrations 915 take the form roughly of chevrons. In FIG. 9B, the absorbent layer 960 of FIG. 9A is coupled (in this case, laminated) to a foam layer 945 to form dressing layer 910. In this Example, the foam layer 945 typically exhibits substantially elastic recovery with the application of a strain, and the fenestrated (915) absorbent layer 960 typically does not exhibit substantially elastic recovery with the application of a strain. Also in this Example, the combination of the coupling of these two layers and the fenestrations 915 are configured to impart substantial elastic recovery to the absorbent layer 960, thus imbuing the dressing layer 910 with substantial elastic recovery with the application of a strain. Also in this Example, the combination of the coupling of these two layers and the fenestrations 915 are configured to impart substantial elastic recovery to the absorbent layer 960, thus imbuing the dressing layer 910 with substantial elastic recovery with the application of a strain in a length direction. Such an applied strain is meant to simulate and/or to approximate the duty of the a wound dressing containing dressing layer 910, when positioned over wound sites in areas of relatively high flexure (particularly knee or elbow joints), for example, thereby eliminating, minimizing, or reducing edema at such wound sites. In addition to dressing layer 910, a typical wound dressing may contain (none of which are shown in FIGS. 9A-9B) at least a backing layer and an adherent layer, for securing the wound dressing to tissue proximate to a wound site.

### Example 8

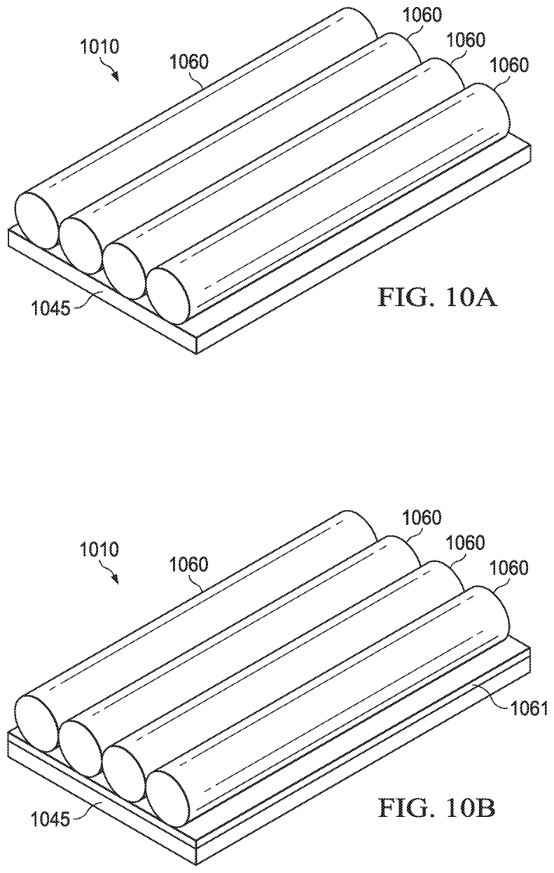

FIGS. 10A-10B show exemplary composite dressing layers 1010. In both FIGS. 10A and 10B, the dressing layer 1010 is a multi-layer composite island comprised of a layer 1060 including an array of cylindrically-shaped bundles of non-woven CMC-based absorbent fibers and an open cell polyurethane foam layer 1045. In FIG. 10A, the cylindrical bundle array layer 1060 is directly coupled (in this case, laminated) to the foam layer 1045. In FIG. 10B, there is an intermediate CMC-based non-woven absorbent layer 1061 disposed therebetween and coupled (in that case, laminated) to both layers. Tn each case, the foam layer 1045 typically exhibits substantially elastic recovery with the application of a strain, and both the cylindrical bundle layer 1060 and absorbent layer 1061 typically do not exhibit substantially elastic recovery with the application of a strain. By virtue of the coupling of these two or three layers together, each composite dressing layer 1010 can exhibit substantially elastic recovery with the application of a strain in a direction approximately perpendicular to the average direction of the cylindrical axes of the bundle array (not shown). The application of a strain is meant to simulate or to approximate the duty of a wound dressing containing composite dressing layer 1010 when positioned over wound sites in areas of relatively high flexure (particularly knee or elbow joints), for example, thereby eliminating, minimizing, or reducing edema at such wound sites. In addition to composite dressing layer 1010, a typical wound dressing may contain (none of which are shown in FIGS. 10A-10B) at least a backing layer and an adherent layer, for securing the wound dressing to tissue proximate to a wound site.

### Non-Limiting Discussion of Terminology

The description and specific examples, while indicating embodiments of the technology, are intended for purposes of illustration only and are not intended to limit the scope of the technology. Moreover, recitation of multiple embodiments having stated features is not necessarily intended to exclude other embodiments having additional features, or other embodiments incorporating different combinations of the stated features. Components may also be combined or eliminated in various configurations for purposes of sale, manufacture, assembly, or use. Specific examples are provided for illustrative purposes of how to make and use the compositions and methods of this technology and, unless explicitly stated otherwise, are not intended to be a representation that given embodiments of this technology have, or have not, been made or tested. Equivalent changes, modifications and variations of some embodiments, materials, compositions and methods can be made within the scope of the present technology, with substantially similar results.

As used herein, the words “include,” “contain,” and their variants, are intended to be non-limiting, such that recitation of items in a list is not necessarily to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments that do not contain those elements or features. Moreover, descriptions of various alternatives using terms such as “or” do not require mutual exclusivity unless clearly required by the context, and the indefinite articles “a” or “an” do not limit the subject to a single instance unless clearly required by the context.

Although the open-ended term “comprising,” as a synonym of non-restrictive terms such as including, containing, or having, is used herein to describe example embodiments of the present technology, embodiments may alternatively be described using more limiting terms such as “consisting of” or “consisting essentially of,” the latter of which has been defined by various judicial interpretations in the U.S. Thus, for any given embodiment reciting materials, components or process steps with such open-ended terms, similar or analogous embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosure of values and ranges of values for specific parameters, such as temperatures, molecular weights, weight percentages, etc., are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, disclosure of two or more ranges of values for a parameter, whether such ranges are nested, overlapping or distinct, may subsume all possible combination of ranges for the value that might be claimed using endpoints of the disclosed ranges. For example, if parameter X is exemplified herein to have values in the range of 1-10, or 2-9, or 3-8, Parameter X may be envisioned as having other ranges of values including 1-2, 1-3, 1-8, 1-9, 2-3, 2-8, 2-10, 3-9, 3-10, 8-9, 8-10, and 9-10.

The term “about,” as used herein, is intended to refer to deviations in a numerical quantity that may result from various circumstances, for example, through measuring or handling procedures in the real world; through inadvertent error in such procedures; through differences in the manufacture, source, or purity of compositions or reagents; from computational or rounding procedures; and other deviations as will be apparent by those of skill in the art from the context of this disclosure. For example, unless otherwise defined by the specification per se or by the context of the specification, the term “about,” with reference to a value, may refer to any number that would round to that value, based on a significant digit analysis. In such a circumstance, a value of “about 30%”, assuming the “3” is the only significant digit, could encompass from 25% to just below 35%. However, the context of the specification would limit that interpretation based on significant digits, so that the “about” ranges do not overlap. For example, if the specification discloses ranges that include “about 25%, about 30%, about 35%,” etc., about 30% in that context could encompass from 27.5% to just below 32.5%. Alternatively, the term “about” may refer to deviations that are greater or lesser than a stated value or range by ±10% of the stated value(s), as appropriate from the context of the disclosure. In such a circumstance, a value of “about 30%” may encompass from 27% to 33%. Whether or not modified by the term “about,” quantitative values recited herein include equivalents to the recited values, for example, deviations from the numerical quantity, as would be recognized as equivalent by a person skilled in the art in view of this disclosure.

The appended claims set forth novel and inventive aspects of the subject matter disclosed and described above, but the claims may also encompass additional subject matter not specifically recited in detail. For example, certain features, elements, or aspects may be omitted from the disclosure and claims, if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention, as defined by the appended claims.

1. A wound dressing comprising:
a composite island configured to collectively exhibit substantially elastic recovery under wound treatment conditions and comprising a foam layer and an absorbent layer coupled to the foam layer, the foam layer exhibiting substantially elastic recovery under wound treatment conditions and the absorbent layer not exhibiting substantially elastic recovery under wound treatment conditions;
a backing layer having a margin that extends beyond the composite island; and
an adherent layer disposed on at least the margin of the backing layer.

2. The wound dressing of claim 1, wherein the adherent layer comprises a hydrocolloid.

3. The wound dressing of claim 1, wherein the absorbent layer comprises:
(i) from about 45% to about 90% of cellulose ether fibers; and
(ii) from about 10% to about 55% of reinforcing fibers.

4. The wound dressing of claim 1, wherein the cellulose ether fibers are composed of at least one of carboxymethyl cellulose, carboxylethyl cellulose, hydroxyethyl cellulose, hydroxypropyl cellulose, and hydroxypropylmethyl cellulose.

5. The wound dressing of claim 1, wherein the foam layer is coupled to the absorbent layer by lamination.

6. The wound dressing of claim 1, wherein the absorbent layer has a plurality of fenestrations collectively configured to mitigate plastic deformation of the absorbent layer.

7. The wound dressing of claim 1, wherein a surface of the absorbent layer not coupled to the foam layer is tissue-facing.

8. The wound dressing of claim 1, wherein a surface of the foam layer not coupled to the absorbent layer is tissue-facing.

9. The wound dressing of claim 1, wherein the absorbent layer coupled to the foam layer is absent in at least a portion of a central zone of its length, such that a surface of the foam layer not coupled to the absorbent layer is tissue-facing.

10. A wound dressing comprising:
a perforated envelope configured to exhibit substantially elastic recovery under wound treatment conditions;
an absorbent layer surrounded by the perforated envelope and having a plurality of fenestrations configured to mitigate plastic deformation of the absorbent layer;
a backing layer having a margin that extends beyond the perforated envelope; and
an adherent layer disposed on at least the margin of the backing layer.

11. The wound dressing of claim 10, wherein the adherent layer comprises a hydrocolloid.

12. The wound dressing of claim 10, wherein the absorbent layer comprises:
(i) from about 45% to about 90% of cellulose ether fibers; and
(ii) from about 10% to about 55% of reinforcing fibers.

13. The wound dressing of claim 10, wherein the cellulose ether fibers are composed of at least one of carboxymethyl cellulose, carboxylethyl cellulose, hydroxyethyl cellulose, hydroxypropyl cellulose, and hydroxypropylmethyl cellulose.

14. A wound dressing comprising:
a composite island configured to collectively exhibit substantially elastic recovery under wound treatment conditions and comprising a foam layer and an absorbent layer coupled to the foam layer, the foam layer exhibiting substantially elastic recovery under wound treatment conditions and the absorbent layer having a plurality of fenestrations collectively configured to mitigate plastic deformation of the absorbent layer and not exhibiting substantially elastic recovery under wound treatment conditions;
a backing layer having a margin that extends beyond the composite island; and
an adherent layer disposed on at least a margin of the backing layer and extending over a margin of the composite island, thereby fastening the adherent layer to the margin of the composite island.

15. The wound dressing of claim 14, wherein the adherent layer comprises a hydrocolloid.

16. The wound dressing of claim 15, wherein the absorbent layer comprises:
(i) from about 45% to about 90% of cellulose ether fibers; and
(ii) from about 10% to about 55% of reinforcing fibers.

17. The wound dressing of claim 16, wherein the cellulose ether fibers are composed of at least one of carboxymethyl cellulose, carboxylethyl cellulose, hydroxyethyl cellulose, hydroxypropyl cellulose, and hydroxypropylmethyl cellulose.

18. The wound dressing of claim 17, further comprising a non-adherent layer.

19. The wound dressing of claim 18, wherein the non-adherent layer is perforated and comprises at least one of an alkyl acrylate polymer, an alkacrylate polymer, and an alkyl alkacrylate polymer.

20. The wound dressing of claim 19, wherein the alkyl acrylate polymer comprises an ethylene-methyl acrylate copolymer.

21. The wound dressing of claim 20, wherein the wound dressing has a width and a length perpendicular to the width, and wherein at least a majority of the plurality of fenestrations are oriented parallel to the width.

22. The wound dressing of claim 21, wherein the absorbent layer further has a plurality of perforations.

23. A wound dressing comprising:
an absorbent layer having a plurality of fenestrations configured to mitigate plastic deformation of the absorbent layer;
a backing layer having a margin that extends beyond the absorbent layer; and
an adherent layer disposed between the absorbent layer and the backing layer and extending onto the margin of the backing layer, thereby forming an adherent layer margin,
wherein each of the plurality of fenestrations extend inward from an edge of the absorbent layer no further than a midpoint between opposing edges, so as to form a central backbone with appendages extending outward therefrom.

24. A wound dressing comprising:
an absorbent layer having a plurality of fenestrations configured to mitigate plastic deformation of the absorbent layer;
a backing layer having a margin that extends beyond the absorbent layer; and
an adherent layer disposed between the absorbent layer and the backing layer and extending onto the margin of the backing layer, thereby forming an adherent layer margin,
wherein at least a majority of the plurality of fenestrations are oriented to form an acute angle between each fenestration and a width or length direction.

25. The wound dressing of claim 24, wherein the backing layer is non-adherent and comprises a polyurethane.

26. The wound dressing of claim 3, wherein the reinforcing fibers are composed of at least one of a polyurethane gel, an amide polymer, an olefin polymer, an ester polymer, and a modified acrylamide polymer.

27. A method of eliminating, minimizing, or reducing edema for a wound surrounded by tissue, the method comprising:
providing a wound dressing having a composite island comprising a foam layer and an absorbent layer coupled to the foam layer, the foam layer exhibiting substantially elastic recovery under wound treatment conditions and the absorbent layer not exhibiting substantially elastic recovery under wound treatment conditions, a backing layer having a margin that extends beyond the composite island, and an adherent layer disposed on at least the margin of the backing layer, and positioning the wound dressing over the wound, such that at least a portion of the adherent layer contacts the tissue.

28. The method of claim 27, wherein the wound is proximal to a knee joint or an elbow joint.

29. The method of claim 27, further comprising:
sealing the wound dressing within a sealed space near the wound; and
applying a negative pressure to the sealed space.

30. (canceled)
